(19) United States
(12) Patent Application Publication
(10) Pub. No.: US 2026/0047867 A1
(43) Pub. Date: Feb. 19, 2026

(54) FIXATION DEVICE FOR TRANSVERSE BONE DISTRACTION

(71) Applicant: ROMAN STAUCH, ASSAMSTADT (DE)

(72) Inventor: ROMAN STAUCH, ASSAMSTADT (DE)

(21) Appl. No.: 19/101,743

(22) PCT Filed: Jul. 4, 2023
(86) PCT No.: PCT/EP2023/068453

§ 371 Date: Feb. 6, 2025

(30) Foreign Application Priority Data
Aug. 10, 2022 (DE) 10 2022 120 185.6

(51) Int. Cl. A61B 17/66; A61B 17/56; A61B 17/64; B33Y 10/00

(52) U.S. Cl. CPC A61B 17/66; A61B 17/6458; B33Y 10/00; A61B 2017/564

(57) ABSTRACT

In a particular embodiment, a fixation device (1) for transverse bone distraction comprising an elongated base (3) having a bone-remote top surface (5), a bone-facing bottom surface (7), a central section (9), a first end (11) and a second end (13), a first outer Schanz screw (15) attached to the first end (11) on the bottom surface (7) of the base (3), and a second outer Schanz screw (17) attached to second end (13) on the bottom surface (7) of the base (3), at least one inner Schanz screw (19) attached to the central section (9) on the bottom surface (7) of the base (3), and at least one displacement device (21) configured to move the at least one inner Schanz screw (19) away from the bone relative to the outer Schanz screws (15, 17).

### FIELD OF THE INVENTION

This patent application relates to a fixation device for transverse bone distraction.

### BACKGROUND OF THE INVENTION

Such devices are used in orthopedics and limb correction, particularly but not exclusively for patients suffering from diabetes. According to the U.S. Centers for Disease Control and Prevention (CDC), approximately 7.8% of the total U.S. population—nearly 24 million people—were diagnosed with diabetes in 2007. These patients often face issues with insufficient blood circulation in the lower legs, which, combined with diabetic preconditions, can lead to severe complications following injuries in this area, up to and including amputation. The treatment of these patients and the complications arising from it generated total indirect and direct costs of $174 billion in the U.S. in 2007.

Peripheral vascular complications and neurological complications closely associated with foot ulcerations accounted for 31% and 24% of these costs, respectively, and were among the primary causes of extended hospital stays. More than 60% of non-traumatic lower-limb amputations occur in diabetic patients, with ulcers preceding at least 80% of these amputations.

In recent years, it has been observed that transverse distraction, i.e., transverse bone distraction via an external fixation device, can significantly improve blood circulation in the lower extremities, thereby generally preventing amputation. This has been confirmed by several studies, including those conducted in China, where over a thousand patients have been treated with such external fixation devices for transverse bone distraction. In this process, a portion of the lower leg bone (tibia) is separated, and new bone tissue is generated by transverse distraction of this segment.

Chinese patent publication CN 106108992 A discloses a fixation device for transverse bone distraction comprising a main body and a parallel secondary body. At their ends, these bodies each comprise receivers for bone pins. The main body and the secondary body are connected via a screw mechanism wherein a knurled nut in the main body can move a threaded rod whose bone-facing end is connected to the center of the secondary body.

However, this known device has several disadvantages. Its design and structure, with numerous adjustment options, make it cumbersome for surgeons to handle, requiring significant time and effort. These extensive adjustment options are unnecessary in the operating room and complicate the use of the device, unnecessarily extending the duration of surgery. Furthermore, the geometry of the known device, with its long protruding pins and screws, poses a risk of the patient getting caught on a table or chair leg or other obstacles with the device attached to their lower leg. Additionally, the structure is inconvenient for daily use, particularly when dressing or sleeping.

### SUMMARY OF THE INVENTION

The objective of the present invention is to at least partially overcome the disadvantages of the prior art by providing a fixation device that is easy to handle, simple in design, lightweight, and facilitates both daily use and application during surgery.

According to an aspect of the invention, a fixation device for transverse bone distraction is provided, comprising an elongated base having a bone-remote top surface, a bone-facing bottom surface, a central section, a first end, and a second end. A first outer Schanz screw is attached to the first end on the bottom surface of the base, a second outer Schanz screw is attached to the second end on the bottom surface of the base, and at least one inner Schanz screw is attached to the central section on the bottom surface of the base. At least one displacement device is configured to move the at least one inner Schanz screw away from the bone relative to the outer Schanz screws, wherein the inner and outer Schanz screws are configured to engage with the bone at their bone-facing ends. In the functional state of the fixation device, the at least one inner Schanz screw, the outer Schanz screws, and the displacement device do not protrude beyond a bone-remote surface of the fixation device, such that the surface of the fixation device forms a substantially flat, projection-free surface.

By providing a flat surface free of protrusions, pikes, points or edges, the device achieves significantly improved handling and reduces the risk of injury to the patient. Additionally, the relatively simple design facilitates operation during surgery. The structure also results in a low overall height and lightweight construction, as it allows for reduced length and width of the device. The term “substantially flat surface” does not refer to a continuous smooth surface; rather, it includes other components integrated into the flat surface, such as the displacement device, that do not impair handling due to protrusions, edges, points, or screws/pins. The transitions between components, such as between the base's top surface and the displacement device, are configured to prevent cables, tubes, clothing, or similar items from snagging or tangling. Consequently, these transitional components may be rounded, beveled, or flattened. The flexible arrangement of the displacement device allows for designs that save installation space, additional components, and thus costs, while enabling a simplified structure.

In embodiments, the displacement device may be configured to a) fix the at least one inner Schanz screw to the base upon actuation; or b) allow the at least one inner Schanz screw to move relative to the base. Embodiment (a) offers a particularly simple, streamlined design, eliminating the need for additional components and simplifying handling for surgeons and medical staff. Embodiment (b) enables precise adjustment of the amount of displacement because only a single element of the displacement device is being adjusted.

In another embodiment, the displacement device may comprise at least one component selected from the group consisting of a knurled or rotating head, a knurled nut, a knurled screw, a screw, a profiled screw head, and a guide element. Other configurations of the displacement device that can be easily operated manually are also conceivable. An adjusting element of the displacement device is intended to cause relative displacement of at least one Schanz screw concerning the base. Adjusting elements with knurled profiles are simple in structure, readily available, and reliable in function. They can be made, for example, of a suitable metal, metal alloy, or plastic such as polypropylene (PP), polycarbonate (PC), acrylonitrile-butadiene-styrene (ABS), polyetheretherketone (PEEK), or another suitable rigid polymer material. In addition to the above, other adjusting elements for relative displacement may also be suitable.

In further embodiments, the fixation device may include at least one fastening device, each configured to prevent or enable displacement of the inner or outer Schanz screws. The fastening device can fix the inner or outer Schanz screws to or relative to the base, ensuring that the fixation device remains in the location specified by the surgeon relative to the patient. This may enhance the healing process since only the bone fragment attached to the variably mounted Schanz screw(s) on the base is moved stepwise away from the bone after initial attachment, allowing new bone tissue to be generated according to the principles of callus distraction. Both the outer and inner Schanz screws can perform the fixing function. In specific embodiments, all Schanz screws may include such a fastening device, for example, to prevent unintended adjustments by the patient or other persons.

In embodiments, the fastening device may comprise a bore on one side of the base and a fastening means accommodated therein. For example, a threaded pin or the like can be inserted into the side bore, which has a corresponding thread, and engage with the Schanz screw in a force-fitting or form-fitting manner via an Allen screw or a similar screw type, preventing movement by clamping it essentially perpendicular to its direction of motion. It should be noted that other clamping mechanisms can also achieve the releasable fixation of the Schanz screws, such as a lever mechanism, tension cone, or eccentric mechanism.

The side bore for the fastening device should not be confused with the vertical bores from the top to the bottom of the base that house the Schanz screws. These vertical bores need not be through-holes. However, a through-hole offers the advantage of allowing the base with through-holes to be used as a drilling jig. This enables precise marking of the insertion points for the Schanz screws on the bone.

In further embodiments, the inner and/or outer Schanz screws may have a metric thread at their upper end and a thread suitable for bone fixation at their lower end. Other types of threads besides metric threads can also be used. The threads at the upper end of the Schanz screws provide a relatively simple displacement mechanism through the displacement device using the aforementioned adjusting elements, such as knurled screws etc. This also enables precise distance adjustment. The threads at the lower end of the Schanz screws are configured to easily penetrate the outer bone tissue and engage with the bone interior. For this purpose, the Schanz screws generally have a sharp or beveled tip that further facilitates bone penetration. Compared to pins without threads, Schanz screws equipped with bone threads provide significantly better adhesion and fixation to the bone.

It should be noted that the Schanz screws do not necessarily need to have a round cross-section at the upper part. For example, the cross-section can be polygonal. They can also be flattened on one or more sides along their length to interact with the displacement device, resulting in torsion-resistant displacement relative to the base. One example of a non-round cross-section at the upper end is flattening on two opposite sides, with the (metric) thread continuing on the remaining outer surfaces. When a nut mounted on the base engages with the Schanz screw and is then turned, the base moves vertically up or down along the Schanz screw, while the Schanz screw remains oriented along its longitudinal axis relative to the base, i.e., there is no rotation of the Schanz screw relative to the base. This effect can also be achieved with other geometric structures of the Schanz screw and the corresponding adjusting elements of the displacement device. It is also conceivable that the Schanz screws in the upper guided area have no threads but only a guiding geometry, i.e., the guidance and threading are separated along the length.

In embodiments, the displacement device may include an adjusting element accommodated in a recess in the base wherein movement of the adjusting element causes displacement of the inner or outer Schanz screws relative to the base. For the application of the fixation device, the distance of the extracted bone fragment from the fixed bone can be adjusted as precisely as possible to optimize treatment. Adjusting elements such as knurled screws with a scale or similar units with appropriate measures are particularly well-suited. Once set, the adjustment cannot change on its own to prevent resetting or backward movement contrary to the distraction. For this purpose, the displacement device can include appropriate friction in the screw or clamping elements to prevent the set distance values from changing on their own. Alternatively, the displacement device can also include additional clamping.

In another embodiment, the base may have at least one recess on its side. This allows for material savings, making the entire fixation device lighter and easier to handle. For example, the recesses can be designed to conform to the contour of a hand. Similarly, the surface of the base can be rounded or chamfered at the edges, including the bone-facing side, to soften any sharp edges further.

In further embodiments, the displacement device may include an elongated inner body, which is preferably receivable in a receiving space on the bottom surface of the base. The at least one inner Schanz screw may be attached to the inner body. In this configuration with at least one inner Schanz screw at the inner body, the above mentioned alternative b) is realized, i.e., actuating the displacement device on the base, to which the outer Schanz screws are fixed, moves the inner body relative to the base. In other words, the movement of the inner body relative to the base leads to a simultaneous and uniform displacement of the inner Schanz screws attached to the inner body. Upon operation of the displacement device, here the whole base does not move together with the at least one inner Schanz screw as in the above mentioned alternative a) but only the inner body.

The displacement device may include a threaded screw which connects the base to the inner body and comprises a knurled head, and which is configured to move the inner body together with the bone segment away from or toward the base by rotation. As described above, this allows for precise adjustments with the most precise possible dimension adjustment. Alternative displacement mechanisms are conceivable which are not based on a thread or a rotation such as Schanz screws with even tops slidably clamped in the area of the base. A ratchet system may be used.

In selected configurations, the base may be at least partially configured as a tubular hollow body. The cross-section may be circular, elliptic, square-shaped, rectangular or polygonal. The displacement device and further elements may be formed as fixedly introduced inserts to ensure proper functionality of the element. Such inserts may, for example, be glued or otherwise fastened to the base.

Alternatively, the base can be constructed from solid materials like metals or alloys. Also, suitable medical-grade plastics may be used, for example thermosetting plastics or thermo-plastics, as well as fiber-reinforced plastics such as GRP (glass-reinforced plastic) or CFRP (carbon fiber-reinforced plastic).

In certain embodiments, at least the base and/or the inner body or other components may be manufactured using 3D printing. 3D printing is a fast and also inexpensive manufacturing process. It is characterized by the fact that different materials can be used on the same work-piece and devices such as the fixation device according to the invention can be manufactured in a single process. Above all, 3D printing may also be used to produce suitable, stable structures like lattice structures, honeycomb structures and others, which are comparatively complex and expensive in conventional manufacturing processes.

Furthermore, the displacement device may be operated with an actuator for precise or automated adjustments. This enables an exact and, where required, automated adjustment of the displacement device. These actuators could be electrically operated while allowing manual overrides for flexibility.

### BRIEF DESCRIPTION OF THE DRAWINGS

The present invention will now be described with reference to preferred examples and embodiments, accompanied by the attached figures, in which:

FIG. 1 shows a perspective view of a first embodiment of the fixation device;

FIG. 2 shows a cross-sectional view and a side view of a portion of the fixation device of FIG. 1;

FIG. 3 shows a perspective detailed view of a second embodiment of the fixation device;

FIG. 4 shows a side view of a Schanz screw according to a preferred embodiment of the fixation device;

FIG. 5 shows a perspective view of a third embodiment of the fixation device;

FIG. 6 shows a schematic detailed view of a displacement device according to the embodiment from FIG. 5;

FIG. 7 shows a side view of a Schanz screw with a part of a displacement device according to another embodiment;

FIG. 8 shows a partial side view of a Schanz screw with a part of a displacement device according to another embodiment; and

FIG. 9 shows a schematic perspective view of a fourth embodiment of the fixation device.

### DETAILED DESCRIPTION OF EMBODIMENTS

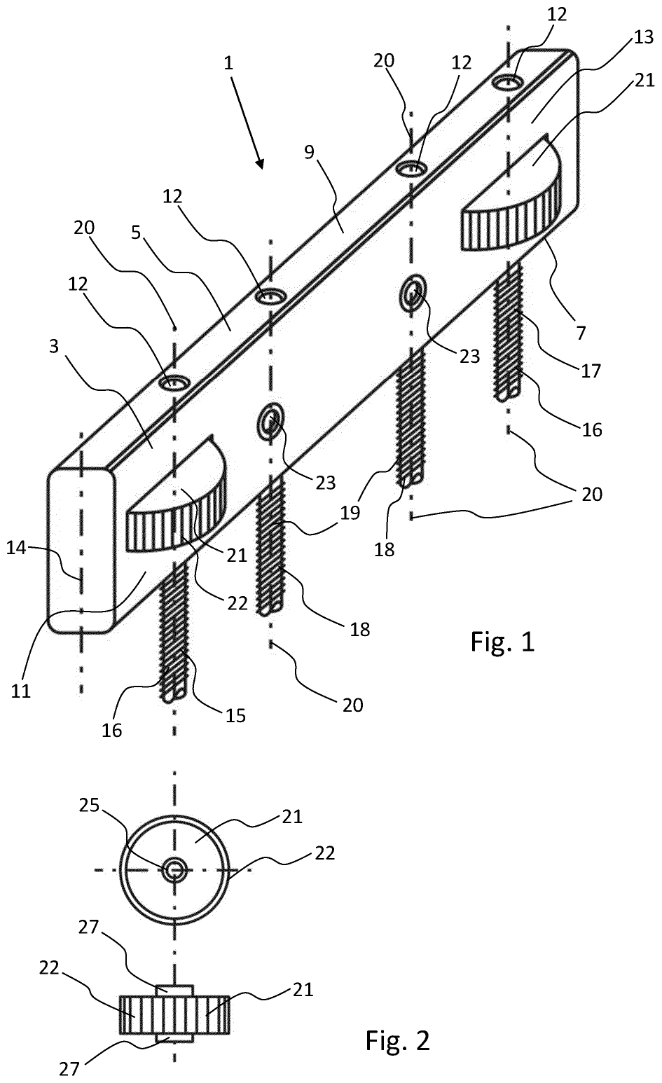

FIG. 1 provides a schematic perspective view of a first embodiment of the fixation device. The fixation device 1 comprises a base 3 which in this embodiment is substantially cuboidal in shape, comprising a top surface 5, a bottom surface 7, a central section 9, a first end 11, and a second end 13. At the first end 11, a first outer Schanz screw 15 is mounted in a through-bore 12 such that it protrudes from the bottom surface 7 of the base 3. Similarly, at the second end 13, another outer Schanz screw 17 is mounted in a through-bore 12 on the bottom surface 7 of the base 3. These two outer Schanz screws 15, 17 are arranged parallel to each other along rotation axes 20 that, in this embodiment, lie in the symmetry plane 14 extending from the top surface 5 to the bottom surface 7 of the base 3.

In the embodiment shown in FIG. 1, the top surface 5 of the base 3 faces away from the bone during transverse distraction, and the bottom surface 7 is directed toward the bone and interacts with the Schanz screws. Central section 9 includes two inner Schanz screws 19 on the bottom surface 7, which are also mounted in bores 12 aligned with the rotation axis 20. These bores 12 for the inner Schanz screws 19 are parallel to each other and, in this embodiment, also parallel to the bores 12 for the outer Schanz screws 15, 17. However, this parallel arrangement is not mandatory. Both the outer and inner Schanz screws are provided with a partial metric (external) thread 18 in the upper region that engages with the base 3. In other words, the upper sections of the Schanz screws not interacting with the displacement device may also be smooth.

The first outer Schanz screw 15 and the second outer Schanz screw 17 are operatively connected to a displacement device 21 in this embodiment. The displacement mechanism 21 is configured to vertically move the base 3, along with the inner Schanz screws 19 attached to it, relative to the outer Schanz screws 15, 17. The displacement device 21 includes an adjustment wheel housed in a recess of base 3. The recess spans from one side to the other of the base 3. It should be noted that this arrangement does not necessarily have to be symmetric and can, for example, be positioned on only one side of base 3. Rotating the adjustment wheel 21 results in axial movement of the base 3 and the attached inner Schanz screws 19, which are fixed to the bone fragment to be distracted, relative to the stationary outer Schanz screws 15, 17. This is achieved by the adjustment wheel 21 featuring a threaded bore 25 at its center, which engages the external thread 16 on the upper sections of the outer Schanz screws 15, 17. The adjustment wheel 21 comprises a knurled surface 22 or a textured grip area around its circumference, configured to allow manual operation, such as with fingers, enabling rotation around the rotation axis 20 of the Schanz screws 15, 17. Consequently, during wheel movement, neither the inner nor the outer Schanz screws rotate around their rotation axes 20.

To ensure that the adjustment wheel 21 is rotatably mounted within base 3 along the rotation axis 20, it preferably comprises a guiding element 27 on its upper and/or bottom surface, as further detailed in FIG. 2. Both inner Schanz screws 19 are securely attached to the base 3 at their upper sections, e.g., by threading or clamping within the bores 12. Once the vertical position of the inner Schanz screws 19 is set, they are preferably secured using a fixation mechanism. In this embodiment, the fixation mechanism consists of a bore 23, substantially perpendicular to the rotation axes 20, with an internal thread that allows for the insertion of a screw or threaded pin to fix the inner Schanz screws 19 axially. However, this fixation mechanism can be adjusted or released during or after the intended use of the fixation device 1. With respect to the fixation mechanism, it is conceivable to prevent its axial movement not by means of a bore with an inserted screw but by means of a different type of clamping. Alternative fixation methods include clamps, wedges, or other securing techniques such as bayonet closures similar to a garden hose clamp.

FIG. 2 shows a cross-sectional and side view of a section of the displacement device from FIG. 1. In the top view, the adjustment wheel 21 is visible, featuring a threaded bore 25 along the rotation axis 20, and a knurled surface 22 around its outer circumference which is configured such that a user may turn the adjustment wheel 21 with her or his fingers. In the side view of FIG. 2, the knurled or grip area 22 is also visible along its circumference. Guiding elements 27 are arranged on the top and/or bottom sides of the adjustment wheel 21, the guiding wheels being configured to enable the adjustment wheel 21 to rotate within the recess of the base 3 substantially with minimal play. It is also possible to provide a proper mount by means of ball or needle bearings. For simplicity of the construction, the embodiment illustrated here avoids complex bearing arrangements. It is also noted that the adjustment wheel 21 can be guided within the base 3 even without guiding elements 27.

The proper functioning of the fixation device in the shown embodiment depends on the outer Schanz screws 15, 17 being securely housed within the bores 12. When the displacement device 21 is activated, e.g., by turning the adjustment wheel, the displacement device 21, along with the base 3 and the attached inner Schanz screws 19, moves vertically relative to the stationary outer Schanz screws 15, 17. Another way to achieve rotationally fixed axial mounting of the outer Schanz screws 15, 17 is to flatten the outer screws in the area where they are housed or guided within the base 3, i.e., within the bores 12. Accordingly, the bores 12 may comprise an inner cross-section complementary to the flattened section of the Schanz screws. It is understood that apart from flattening other geometric designs of the outer Schanz screws are also possible. Similarly, alternative shapes and counterparts for the displacement device 21 and the upper ends of the outer Schanz screws 15, 17 are feasible. Base 3 can thus be rotationally fixed relative to the outer Schanz screws 15, 17, ensuring that the screws cannot rotate axially but only move vertically along their longitudinal axis, driven by the rotation of the displacement device 21.

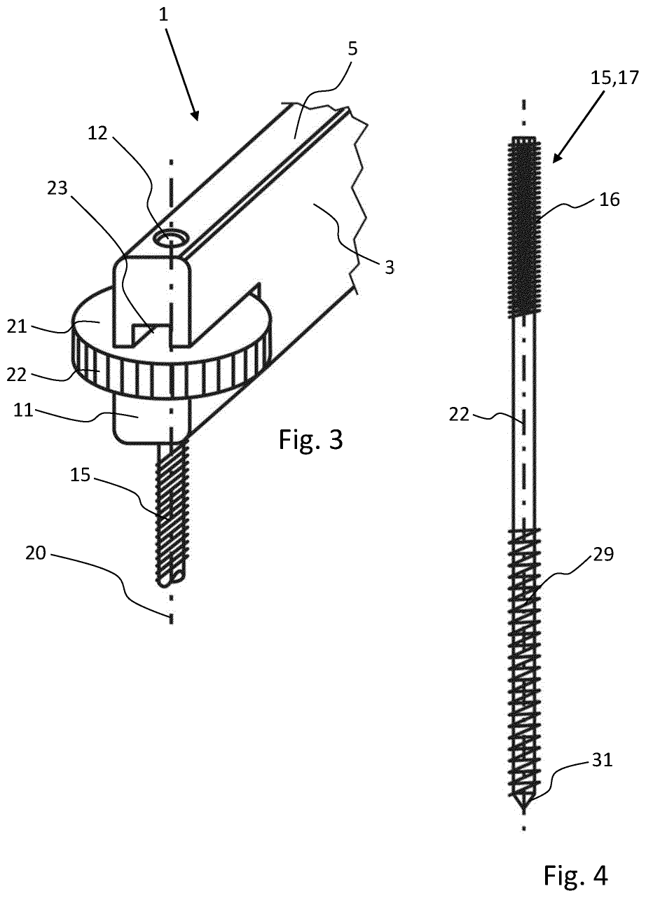

FIG. 3 presents a perspective detailed view of a second embodiment. In contrast to the first embodiment shown in FIG. 1, the outer Schanz screws 15, 17 in the second embodiment are positioned at the extreme ends of the base 3. The configuration and attachment of the inner Schanz screws 19 remain largely identical and are therefore not depicted in FIG. 3. At the first end 11, bore 12 with an inserted outer Schanz screw 15 is positioned at a slight distance from the face of the base 3. The adjustment wheel 21 retains the same internal configuration as in the first embodiment but extends beyond the face of base 3. In base 3 the recess accommodating the adjustment wheel 21 is enlarged such that the adjustment wheel 21 with the guiding elements 27 (not shown in FIG. 2) may be inserted from the face of the base 3. This recess can also be used to house a fixation device 23 that prevents axial rotation of the adjustment wheel 21. For example, a threaded pin or clamp element which is introduced from the face or the side can be inserted to secure the adjustment wheel 21 between the base 3 and its surface. Other configurations of the clamping device are also possible. An advantage of the second embodiment shown in FIG. 3 is that the base can be configured to be shorter, and the contact surface or the circumferential surface of the adjustment wheel 21 is larger in relation to the first embodiment is enlarged. This improves handling somewhat. In addition, for example, the readability of a measuring scale applied to the circumferential surface of the adjustment wheel 21 may be enhanced facilitating to adjust the axial displacement of the outer Schanz screws 15, 17 as precisely as possible.

FIG. 4 illustrates a side view of a Schanz screw in another embodiment. The Schanz screw used here is typically made of a medically compatible plastic, metal, or an appropriate metal alloy. Its upper section comprises a thread 16, a largely smooth middle section, and a so-called bone screw thread 29 in the lower section. As mentioned earlier, the material may also be a plastic that meets medical compatibility and usability requirements in transverse bone distraction. It is understood that the Schanz screws only need to comprise a thread in their upper section if they engage with a displacement device having an internal thread to enable axial movement. Consequently, the upper sections of the Schanz screws may also be threadless. The bone screw thread 29 has a relatively high thread pitch to allow the Schanz screw to be driven into the bone with as few turns as possible. For precise placement and ease of drilling into the bone surface, the Schanz screw preferably has a beveled, sharp tip at its lower end. The thread 16 in the upper section of the screw is a metric thread in the embodiments presented here, for example, with an external diameter of 4 mm. Alternatively, other thread types, such as an inch-based thread, may be used. The thread lengths are adapted to the type of bone being distracted and the extent of the distraction. For example, distraction of the tibia typically requires a distraction range between 5 mm and 25 mm, preferably between 10 mm and 20 mm. As mentioned earlier, the cross-section of the Schanz screw in the upper section need not necessarily be circular. It may be non-circular or asymmetrical to ensure rotational stability. For instance, the Schanz screw may be flattened on two opposing sides or have a D-profile, i.e., smooth on one side and threaded around the remaining circumference.

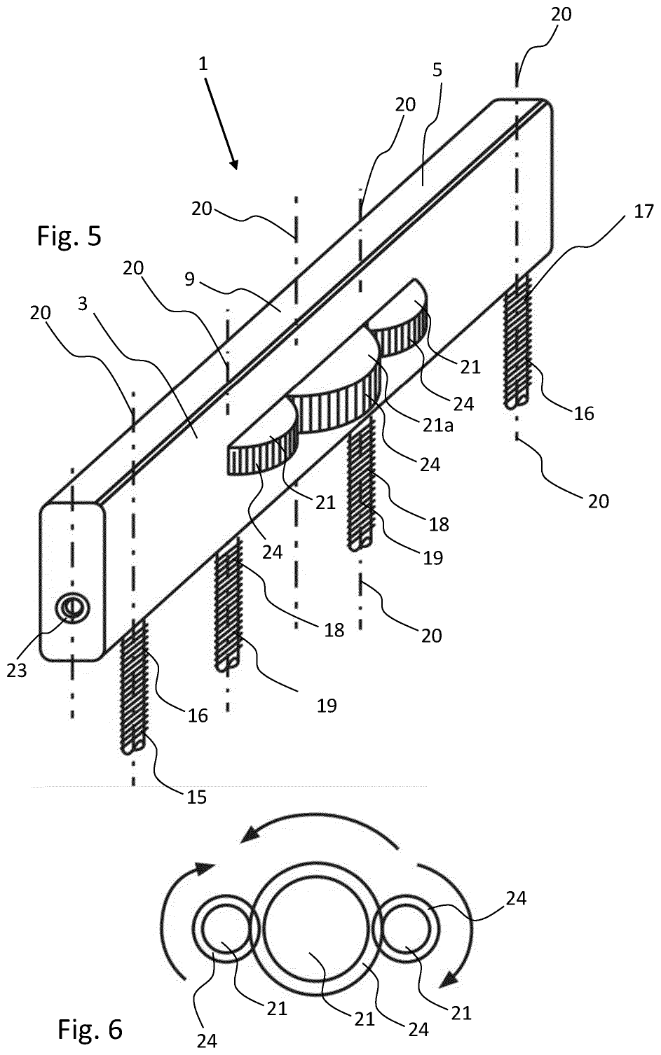

FIG. 5 presents a schematic perspective view of a third embodiment. The basic structure of the fixation device 1 in this embodiment is similar to the first embodiment in FIG. 1, especially with the base 3 having essentially the same shape. Therefore, the description of this embodiment will primarily focus on differences from the first embodiment. The first outer Schanz screw 15 is also attached at the first end 11 of the base 3, and the second outer Schanz screw 17 is similarly attached at the second end 13 of the base 3, inserted from the bottom surface 7 into bores 12, which may or may not extend through the top surface 5 of the base 3 although not visible in the shown perspective view. In this embodiment, the bores 12 do not necessarily pass through, creating a blind hole that results in a smooth and flat top surface 5 of the base 3 without any recesses or protrusions. The first outer Schanz screw 15 and the second outer Schanz screw 17 are received in the bores 12 and are each fixed or held in place by a fastening device 23. Similar to the description of the fastening device 23 in relation to the first embodiment in FIG. 1, the fastening device 23 here comprises a threaded bore which extends from the end face of the base 3 parallel to the longitudinal axis of the base 3 into the bore 12. For example, a grub screw with a hexagon socket is inserted into this threaded hole so that it can be screwed in or unscrewed by an appropriate tool or clamped from above (see FIG. 8). The second outer Schanz screw 17 is also fixed in the same way by a fastening device 23 (not visible here). Here it is also possible that the hole belonging to the fastening device 23 is not arranged on the front side, but on the side surface of the base 3.

Accordingly, in this third embodiment in FIG. 5, the inner Schanz screws 19 are configured so that they can be displaced relative to the base 3 in their longitudinal direction along the axes 20 by the displacement device 21, namely by means of a central adjustment wheel 21a which is arranged between two substantially identical adjustment wheels and interacts therewith. In this third embodiment, the displacement device 21 comprises a respective adjustment wheel which, as in the first embodiment, is aligned coaxially with the corresponding Schanz screw and is arranged substantially centrally with respect to the axis of symmetry 14 within the base 3. As described with reference to FIG. 1, a rotation of the adjustment wheel causes a longitudinal displacement of the inner Schanz screw 19 along the axis 20, wherein the Schanz screw 19 does not rotate during the displacement about the axis 20, but remains arranged in a rotationally fixed manner. The arrangement of the second inner Schanz screw 19 is identical to the first Schanz screw 19 including the adjustment wheel. The rotation of the two adjustment wheels, and thus the vertical displacement of the two inner Schanz screws 19, is caused by the rotation of the adjustment wheel 21a. The adjustment wheel 21a comprises a toothing 24 on its circumferential surface which meshes or engages with the toothing 24 on the circumferential surface of the two adjacent adjustment wheels. The adjustment wheel 21a, which is depicted slightly larger here, is also arranged centrally within the base 3 in relation to the plane of symmetry 14 and can also comprise guide elements 27 (not visible here) which serve to ensure that the adjustment wheel 21a can move and rotate sufficiently within the base 3. The two outer adjustment wheels each engage with their toothing 24 in that of the adjustment wheel 21a and, due to the geometry of exactly the same size, cause a synchronous vertical displacement of the two inner Schanz screws 19 in relation to the base 3. In particular, the central adjustment wheel 21a may comprise a measure or scale on its top surface or circumference, on which the value of the vertical displacement of the two inner shear bolts 19 may be read. This allows medical personnel or even the patient himself to read or set the value of the transverse distraction. It is understood that other simple adjustment mechanisms can also be included in the displacement device 21 which achieve the same effect, namely the synchronous vertical displacement of the inner Schanz screws 19.

In a further embodiment similar to the embodiment of FIG. 5, the central adjustment wheel 21a can also be arranged in a different plane to the adjustment wheels which are axially aligned with the inner Schanz screws 19. For this purpose, the adjustment wheel 21a may comprise a toothed shaft inside the base 3, for example, which engages in the toothing of the two adjustment wheels. In this way, a transmission ratio can be achieved, making the displacement device smoother and more precisely adjustable.

FIG. 6 provides a schematic detailed view of components of the displacement device 21 according to the third embodiment shown in FIG. 5. Rotating the central adjustment wheel 21a counterclockwise results in synchronized, opposite-direction rotations of the outer spur or adjustment wheels, the toothing 24 of which engages with the circumferential toothing 24 of the adjustment wheel 21a. Clockwise rotation of the central adjustment wheel 21a produces corresponding counterclockwise movements in the spur wheels.

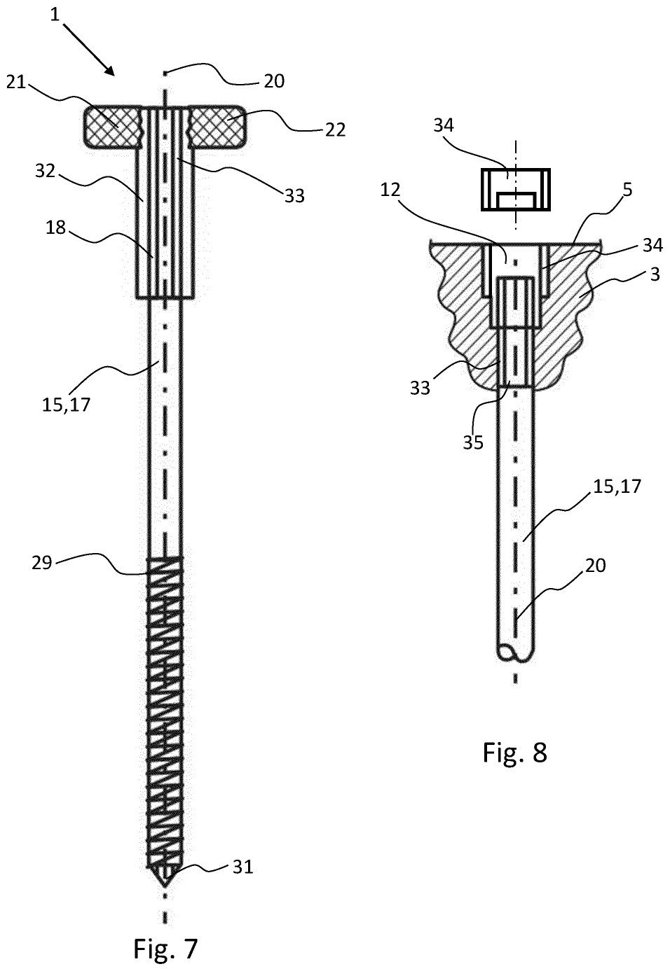

FIG. 7 depicts a side view of a Schanz screw with a part of a displacement device according to another embodiment of the fixation device. As described in earlier embodiments, the axially displaceable Schanz screws may be positioned either at the central section 9 or at the outer ends 11, 13 of the base. The Schanz screw 15, 17 shown in FIG. 7, along with its displacement device 21, may for example each be used as an outer Schanz screw in the first embodiment depicted in FIG. 1.

In contrast to the first embodiment, the adjustment wheel of the displacement device 21 comprises a shaft 32 in the center which extends from the bone-facing bottom surface of the adjustment wheel and is received in the correspondingly larger bore 12 in the base 3 such that smooth rotation and respective guidance is enabled. As in the previously described embodiments, the circumferential surface of the adjustment wheel 21 has a knurling so that an operator can move or rotate the displacement device 21 and thereby cause a displacement of the base 3 relative to the Schanz screws 15, 17. The displacement device 21 is rotatably attached to the base 3 by a corresponding guide element in such a way that it can rotate with respect to the axis of rotation 20, but cannot be displaced in the vertical direction, i.e. along the axis of rotation 20. The rotation of the displacement device 21 thus ensures a corresponding vertical displacement of the base 3 with respect to the Schanz screw 15, 17.

With regard to the flat top surface of the adjustment wheel of the displacement device 21, it should be noted that this is flush with the surface or top surface 5 of the base 3 so that the entire surface of the fixation device 1 remains free of protrusions and essentially flat. If the top surface 5 of the base 3 is not even, the contour of the top surface of the adjustment wheel is adapted accordingly so that the transition between the components is essentially seamless and without an edge.

FIG. 8 provides a partial side view of a Schanz screw within a bore in the base in another embodiment of the fixation device according to the invention. In this embodiment, bore 12 which in the first embodiment passes entirely through base 3, includes a widened upper section near the top surface 5. This widened section is configured to accommodate a clamping or locking element 34. This clamping or locking element 34 shall be adapted to fix the Schanz screw 19 within the base 3. For example, it comprises a cover shape with an external thread that can be engaged with the internal thread of the enlarged hole 12, and also an internal surface that engages with the upper end of the Schanz screw 19 and clamps or fixes it. This inner surface may be beveled, for example, and the upper surface of the screw or clamping element 34 may comprise an internal hexagon socket so that the element can be inserted into the upper portion of the bore 12 for clamping and fixed there. It is understood that other clamping mechanisms can be used here, which are adapted to fix the Schanz screw 19 vertically within the bore 12 of the base 3 without a component of this fastening device projecting beyond the top surface 5 of the base 3. Rather, it is advantageous if the top surface of the screw or clamping element 34 is flush with the top surface 5 of the base 3.

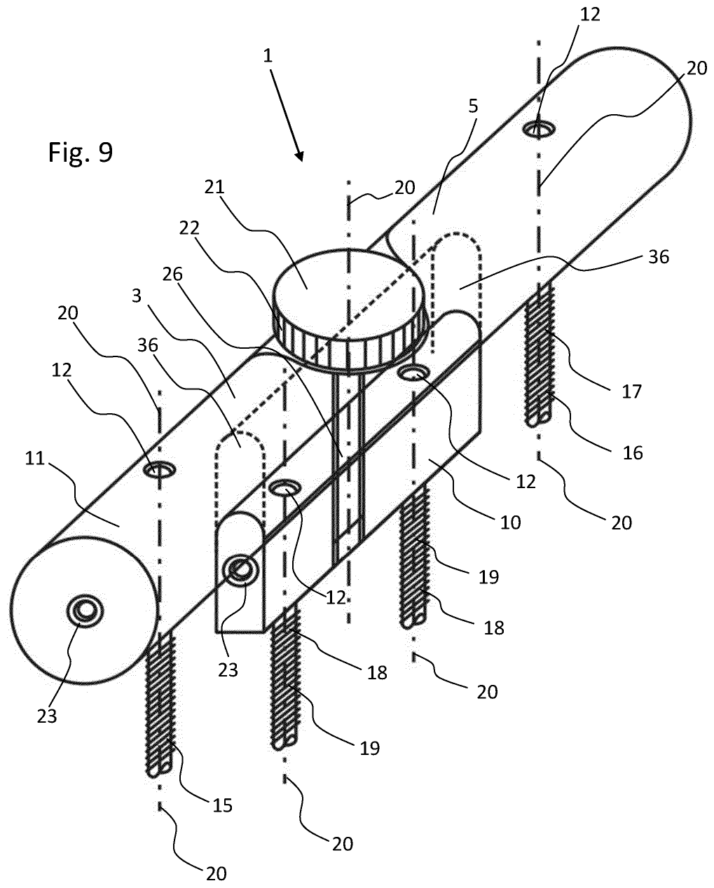

FIG. 9 shows a schematic perspective view of a fourth embodiment of the fixation device. Unlike earlier embodiments, this version includes a cylindrical base 3 with a round or rounded top 5 and an bottom surface 7. The arrangement of the inner and outer Schanz screws is similar to the third embodiment shown in FIG. 5. I.e., the outer Schanz screws 15, 17 are fixed vertically in the respective holes 12, here by means of the fastening device 23, which is arranged on the end face of the base 3 and here represents a threaded hole into which a clamping screw is inserted for clamping the Schanz screw 15. As already described, the fastening device 23 can also be arranged on the side of the base 3 and correspondingly on the inside.

However, the two inner Schanz screws 19 here are not directly attached to base 3, but to an inner body 10, which comprises an elongated, cuboid-like shape and is received on the bottom surface 7 of the base 3 in a receiving space 36 of the base 3. The receiving space 36 is preferably configured in such a way that the contour of the inner body 10 is received therein as precisely as possible, i.e. without much room for movement, and can be moved vertically parallel to the direction of the outer Schanz screws 15, 17. The inner body 10 is preferably made of the same material as base 3. The inner Schanz screws 19 are attached to the inner body 10 in a similar way as the outer Schanz screws 15, 17 are attached to the base 3, i.e. in the functional state they are no longer rotatable relative to the inner body 10 and are fixed vertically with respect to the inner body 10 by means of a fastening device 23.

A displacement device 21 provides the interaction, i.e. the relative displacement, of base 3 with the inner body 10 and thus the inner Schanz screws 19 arranged thereon. A screw 26 is mounted in a bore which is arranged substantially parallel to the other bores 12 approximately in the center of the base 3, which screw 26 comprises an external thread which engages with a threaded bore approximately in the center of the inner body 10. Screw 26 causes a vertical displacement of the inner body 10 with respect to the base 3 upon rotation. With respect to the base 3, the screw 26, which can be formed integrally with the displacement device 21, is rotatable within the bore. The displacement device 21 is attached to the base 3 by a corresponding guide element in such a way that the displacement device 21 can rotate with respect to the axis of rotation 20, but cannot be displaced in the vertical direction. A rotation of the adjustment wheel of the displacement device 21, for example by an operator on the circumferential surface provided with knurling 22, causes a vertical displacement of the inner body 10 and thus of the inner Schanz screws 19. In this embodiment, the maximum bone distraction path is determined by the distance by which the inner body 10 can be moved within the receiving space 36.

It should be noted that the illustration in FIG. 9 is purely schematic in order to explain the interaction of the displacement device 21 with the base or inner body. As described above, the top surface of the displacement device 21 is configured in such a way that it is flush with the top surface 5 of the base 3, wherein this transition is configured without edges or protrusions. Since, in the embodiment shown here, the displacement device 21 is provided with an adjustment wheel, the top surface 5 of the base 3 must have a corresponding recess and be flattened so that the entire surface of the fixation device 1 forms an essentially flat, projection-free surface. In this fourth embodiment, the adjustment wheel 21 of the displacement device may also comprise a scale on its circumferential surface, which serves to measure and adjust the displacement of the inner Schanz screws 19 with respect to the base 3.

As an alternative to mounting the displacement device 21 on the top surface 5 of the base 3, the displacement device 21 can also be arranged inside the base 3, as shown, for example, in the first embodiment in FIG. 1 for the displacement device of the outer Schanz screws 15, 17.

Other possible configurations and embodiments of the fixation device 1 relate to the design of the base 3, which can be designed as a hollow body, for example. In this case, corresponding inserts, which are firmly connected to the base 3, perform the fastening tasks for the inner and outer Schanz screws and the displacement device(s).

This invention thus provides a fixation device that is user-friendly, lightweight, simple in design, and simplifies application during surgery and daily use.

### LIST OF REFERENCE NUMBERS

1 fixation device
3 base
5 top surface
7 bottom surface
9 central section
10 inner body
11 first end
12 bore
13 second end
14 symmetry axis
15 first outer Schanz screw
16 thread
17 second outer Schanz screw
18 thread
19 inner Schanz screw
20 rotation axis
21 displacement device
21a adjustment wheel
22 knurl
23 fastening device
24 toothing
25 threaded bore
26 screw
27 guiding element
29 bone screw thread
31 tip
32 shaft
33 internal thread
34 screw or clamping element
36 receiving space

1-14. (canceled)

15. Fixation device for transverse bone distraction, comprising
an elongated base having a bone-remote top surface, a bone-facing bottom surface, a central section, a first end and a second end,
a first outer Schanz screw attached to the first end on the bottom surface of the base, and a second outer Schanz screw attached to the second end on the bottom surface of the base,
at least one inner Schanz screw attached to the central section on the bottom surface of the base, and
at least one displacement device configured to move the at least one inner Schanz screw away from the bone relative to the outer Schanz screws,
the inner and outer Schanz screws being configured to engage with the bone at their bone-facing ends,
and wherein, in a functional state of the fixation device, the at least one inner Schanz screw, the outer Schanz screws and the displacement device do not protrude beyond a bone-remote surface of the fixation device, such that the surface of the fixation device forms a substantially flat, projection-free surface.

16. The fixation device of claim 15, wherein when the displacement device is actuated,
a) the at least one inner Schanz screw is fixed to the base, or
b) the at least one inner Schanz screw is movable relative to the base.

17. The fixation device of claim 15, wherein the displacement device comprises at least one component of the group consisting of a knurled or rotating head, a profiled screw head, a knurled nut, a knurled screw, a screw and a guide element.

18. The fixation device of claim 15 comprising at least one fastening device, each fastening device being configured to prevent or to enable a displacement of the inner Schanz screws or the outer Schanz screws, respectively.

19. The fixation device of claim 18, wherein the fastening device comprises a bore on one side of the base and a fastening means accommodated therein.

20. The fixation device of claim 15, wherein the inner and/or outer Schanz screws comprise a metric thread at their upper end and a suitable thread for bone fixation at their lower end.

21. The fixation device of one claim 15, wherein the displacement device comprises an adjusting element accommodated in a recess in the base wherein a movement of the adjusting element causes a displacement of the at least one inner Schanz screw relative to the outer Schanz screws.

22. The fixation device of claim 15, wherein the base comprises at least one recess on the side.

23. The fixation device of claim 15, wherein the displacement device comprises an elongated inner body.

24. The fixation device of claim 23, the inner body being receivable in a receiving space on the bottom surface of the base.

25. The fixation device of claim 24, wherein the at least one inner Schanz screw is attached to the inner body.

26. The fixation device of claim 23, wherein the displacement device comprises a threaded screw with a head, the threaded screw connecting the base to the inner body and being configured to move the inner body relative to the base towards or away from the bone by rotation.

27. The fixation device of claim 15, wherein the base is at least partially configured as a tubular hollow body.

28. The fixation device of claim 15, wherein at least the base is produced by means of 3D printing.

29. The fixation device of claim 15, wherein the displacement device is operable by means of an actuator, preferably electrically.

30. The fixation device of claim 29, wherein the actuator is an electric actuator.
